(19) United States
(12) Patent Application Publication
(10) Pub. No.: US 2004/0158156 A1
(43) Pub. Date: Aug. 12, 2004

(54) Liquid crystal thermal imaging of equine lameness

(75) Inventors: Lynn Frances Schneemeyer, Westfield, NJ (US); Joan Corinne Bachenko, Oxford, NJ (US)

Correspondence Address: William K. Hagmann, 871 Shackamaxon Drive, Westfield, NJ, 07090, US

(73) Assignee: Thermal View Wraps LLC

(21) Appl. No.: 10/364,802

(22) Filed: Feb. 11, 2003

(51) Int. Cl. A61B 5/00; A61B 6/00

(52) U.S. Cl. 600/474; 600/549

(57) ABSTRACT

The present invention relates to a device for assessing localized heat resulting from underlying injury in horses, or other mammals, through thermal imaging employing thermochromic liquid crystal displays and methods for their use. The device of the present invention comprises a wrap made of flexible fabric, and incorporates a sheet or sheets of thermochromic liquid crystal material as “windows”, held securely against possible injury sites. A method is claimed of detecting underlying injury in mammals by (a) applying a thermal imaging wrap to an area that is suspected of harboring an injury, (b) applying a second thermal imaging wrap in a parallel or bilateral region not suspected of harboring an injury, and comparing the results of (a) and (b). Besides horses, other mammals, such as dogs, donkeys, mules, llamas, camels, and humans may likewise be evaluated.

The present invention relates to a device for assessing localized heat resulting from underlying injury in horses, or other mammals, through thermal imaging employing thermochromic liquid crystal displays and methods for their use.

### BACKGROUND OF THE INVENTION

Personal and commercial horse ownership has been a popular recreational pastime and a lucrative business for many years. Although all horse owners must provide for the care, feeding, and well being of their horses, they must also be constantly aware of injury and soreness. The owners have complete responsibility in maintaining their horses in an injury-free condition especially with respect to lameness. Therefore, it is vital to keep their charges free from injury in order for the horses to be able to perform at their optimum level in everyday activities or in races.

Horses legs, especially the forelegs, are the most common sites for injuries. Horses carry about 65% of their weight on their forelegs. These front legs absorb the majority of the concussive forces of their hooves hitting the ground. This impact can and often does result in soft tissue injuries. Many of these leg injuries occur when horse are trained too hard, too fast, or on a hard surface. Rapid and hard training does not allow enough time for bones, muscles, ligaments, and tendons to adjust to additional stress. Injury to legs can also occur if the horses have poor conformation, are worked on an uneven surface, or lack balanced hoof trimming and proper shoeing. Similarly, soft tissue injuries to other parts of the animal, such as the back and neck, can also result in poor performance.

As a result, trainers as well as horse owners use thermal imaging techniques to assess the horse's soundness with respect to lameness. In its most basic application, an experienced horse owner or trainer may assess a possible injury by feeling the animal's body, such as the tendon area of a suspected leg, and comparing whether it “feels” warmer than the leg on the other side. Such assessment by feeling is highly variable and subjective. Generally, human hands cannot distinguish between temperature differences of about 5 degrees or less. Alternatively, ultrasound is recognized by the veterinary profession as a useful diagnostic tool for assessing underlying causes of a horse's lameness. [V. B. Reef, “Ultrasonograpy”, World Equine Veterinary Review, 4 (3), 14, 1999]. Likewise, thermal imaging techniques employing infrared cameras and imaging software have also proved useful in assessing lameness. [T. A. Turner, R. C. Purohit, J. F. Fessler, “Thermography: A review in equine medicine”, Comp. Cont. Ed. 8, 855, 1986].

Whereas manual assessment is always a ready, inexpensive and time honored option, the latter two techniques of ultrasound and thermal imaging with infrared cameras require expensive equipment and specialized training. As such, only commercial horse owners with significant investments in their horses are likely to invest further expense in ultrasound or infrared camera thermal imaging techniques to accurately assess injury in their horse. The recreational or small-time horse owner is less likely to invest in or pay for expensive equipment to be employed in accurately assessing their horse's condition, possibly deferring proper treatment and potentially exacerbating the injury by riding and working their horses prematurely. Rather, a veterinarian would be employed on a limited number of occasions to examine and assess the injured area.

Boots are ubiquitous among the horse community and are used, for example, to prevent cuts, to provide tendon support during exercise, and to keep biting insects away. Likewise, horse owners also use neck wraps to keep the mane in place or to help improve the appearance of the neck. Hock wraps are used to give protection to injured hocks. Protective saddle pads are used to ease stress on the horse's back. None of these provides information about underlying heat due to inflammation in the affected area.

Thermochromic materials change color with temperature. Such materials are described in the “Handbook of Thermochromic Liquid Crystal Technology”, Hallcrest, pp. 1-36 (1991). Related art of interest that employs thermochromic materials for the visual display of temperature changes may be found, such as dermally applied thermometers and “mood rings”, but none describes the present invention as it relates to the diagnosis of underlying injury in mammals, esp. horses.

U.S. Pat. No. 5,202,677 issued on Apr. 13, 1993 to Crystal Images, Inc. describes a visual display apparatus using thermochromic materials.

U.S. Pat. No. 5,223,958 issued on Jun. 29, 1993 to Hyperdesign, Inc. describes a heat activated device stimulated by the transfer of thermal energy from a person's body to reveal by color differentiation a hidden message or visual image.

U.S. Pat. No. 5,484,205 issued on Jan. 16, 1996 to Asulab S. A. describes a temperature indicator and watch to determine a person's temperature.

U.S. Pat. No. 5,678,566 issued on Oct. 21, 1997 to Diagnostic Thermographics, Inc. describes a method and apparatus for the diagnosis of abnormalities of the plantar surface of the foot employing an insole fabricated with thermochromic materials.

### SUMMARY OF THE INVENTION

The device of the present invention comprises a wrap made of flexible fabric, and incorporating a sheet or sheets of thermochromic liquid crystal material as “windows”, held securely against possible injury sites. Thermochromic is defined as changing color in response to changes in or gradients of heat. Such materials are described in the Handbook of Thermochromic Liquid Crystal Technology, Hallcrest, pp. 1-36 (1991). The device is designed to remain securely in place in good thermal contact with the animal's body, held in place by straps, typically hook and loop type fasters such as Velcro®, to ensure a proper assessment of the temperature distribution in an injured area in a comparison between injured and healthy areas on the two sides of a horse's body. The underlying heat of an injured area would produce color changes in the liquid crystal material that would be distinguished from the color pattern displayed in corresponding normal, healthy areas on the other side of the animal's body. The device of the present invention uses a flexible fabric with insets of pieces or sheets of temperature sensitive liquid crystal material. The liquid crystal sheet material must be positioned over the site of the injury. Like expensive thermal imaging techniques, the liquid crystal display material will be sensitive to the temperature of the surface of the horse's skin which, in turn, will be a reflection of underlying heat resulting from injury.

A method is claimed of detecting underlying injury in horses comprising (a) applying a thermal imaging wrap to the horse in an area that is suspected of harboring an injury, (b) applying a second thermal imaging wrap to the horse in a parallel or bilateral region not suspected of harboring an injury, and comparing the results of (a) and (b). In addition to injury to legs, such devices may be used to detect heat in feet, hocks, backs, stifles, fetlocks, pasterns, neck, and shoulders. Besides horses, other mammals, such as dogs, donkeys, mules, llamas, camels, and humans may be likewise evaluated.

### BRIEF DESCRIPTION OF THE DRAWINGS

The objects of the present invention will be described with respect to the following figures in which

FIG. 1 is a side view of an open front leg tendon thermal imaging wrap.

FIG. 2 is a side view of an open hock thermal imaging wrap.

FIG. 3 is side view of an open fetlock thermal imaging wrap.

FIG. 4 is an illustration of the placement of thermal imaging wraps on potentially injured areas of a horse.

### DETAILED DESCRIPTION OF THE INVENTION

A device is described which is useful in assessing sub acute or chronic injury of a leg or other area of a horse, or other mammal through the use of a thermochromic liquid crystal display. In one embodiment of the present invention might best be described as a wrap attached around a horse's leg, or other injured part that has incorporated pieces or sheets of thermochromic liquid crystal material which will change color in response to heat arising from underlying injury relative to a parallel or bilateral uninjured area. It should be borne in mind that the present invention need not be limited in use for horses in general, but may find wide application with other mammals, including humans.

The thermochromic material consists of a flexible transparent plastic layer that has been coated with a layer of liquid crystal material chosen so that the color change will be observed in a desired temperature range. A layer of black matte paint is coated over the liquid crystal layer to provide contrast. Sheets of thermochromic material are commercially available from several companies, including Hallcrest, Inc. (Glenview, Ill.) and Color Change Corp. (Addison, Ill.). They are available with a variety of distinct start temperatures, temperature bandwidths, and clearing temperatures. A start temperature is defined as the temperature at which the thermochromic material begins to change color from black to red. The clearing temperature is defined as the temperature at which the thermochromic material changes from blue to black. The difference between the start and clearing temperatures is defined as the bandwidth.

A gradient of temperature change will result in a gradient of colors, giving a rainbow-like appearance. For devices of the present invention, a start temperature of 20 degrees Centigrade and a bandwidth of 10 degrees Centigrade is preferred. The sensitivity of the thermochromic material of the present invention will allow for the discernment of temperature changes of less than 1 degree Centigrade.

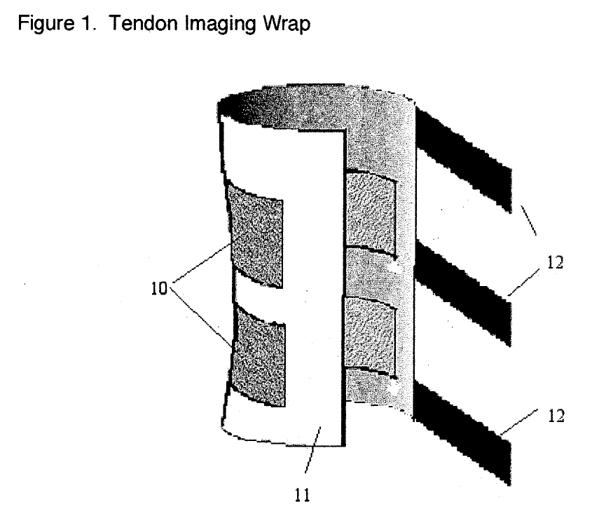

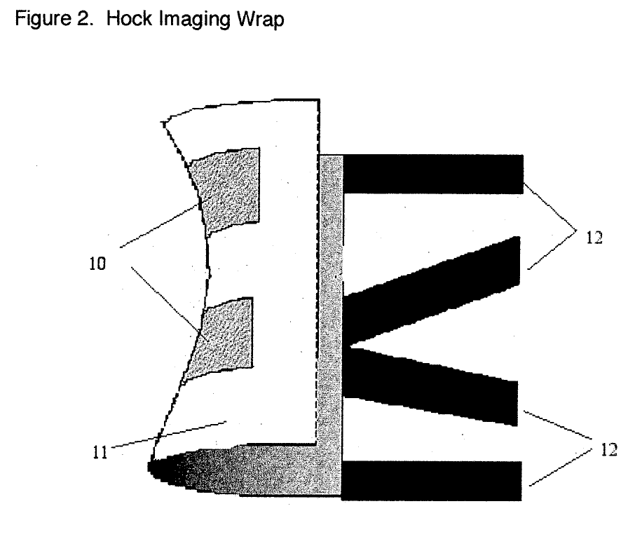

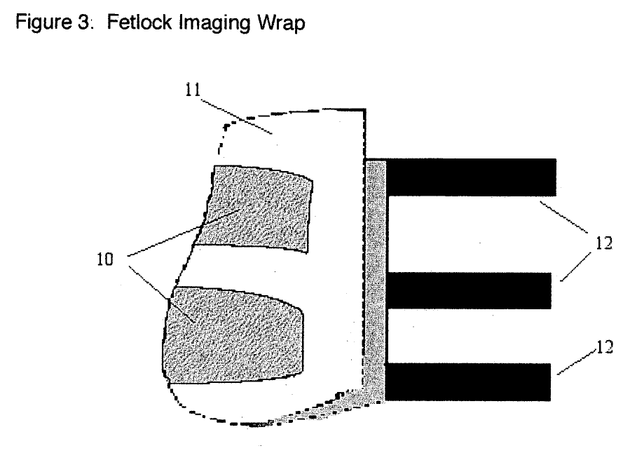

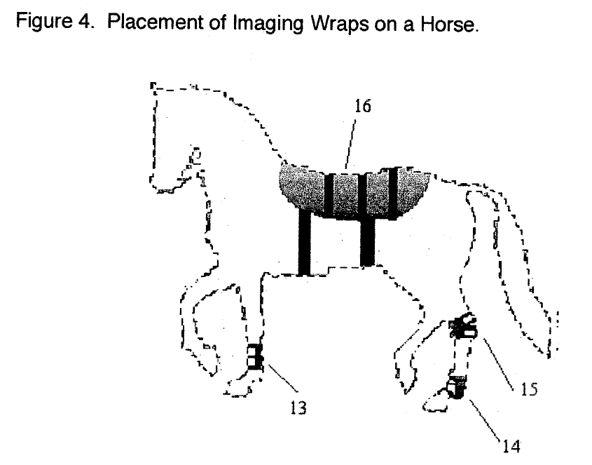

FIGS. 1-3 illustrate the preferred embodiments of the present invention. The wraps are comprised of flexible Velcro® loop fabric 11 incorporating windows 10, preferably made of flexible plastic such as polyester (Mylar), coated with encapsulated liquid crystals and having a black background, normally a matte black paint layer, behind the liquid crystal layer so that the temperature-associated color may easily be observed. These wraps may also be comprised of other materials, such as heavy cloth, normally a dark color that provides ready observation of the temperature-associated color, impregnated or painted with thermochromic liquid crystals. The liquid crystals sheets are incorporated as strips or “windows” into a flexible fabric which is held firmly in place by straps or hook and loop-type fasteners 12. The wraps allow real time visual assessment and evaluation of the heat distribution in an area of the body. Observation of localized heat in only one part of an animal's body suggests underlying inflammation in that area. Although the preferred embodiments illustrate thermal imaging wraps shaped to particular body parts, in this instance as shown in FIG. 4, the tendon of the lower front leg 13, fetlock joint 14, the hock joint 15, and the back 16, there are other shapes of wraps that may be used for thermal imaging of other parts of an animal's body.

What is claimed:

1. An apparatus for aiding in the observation of sub acute and chronic conditions of various parts of the body of a horse or other mammal that produce localized heat due to underlying injury and inflammation; said apparatus comprising a wrap of flexible fabric having insets of thermochromic liquid crystal material which is shaped to conform tightly to a part of the animal's body; and is held tightly in place by straps.

2. An apparatus according to claim 1 which is held tightly in place by hook and loop fasteners such as Velcro®.

3. An apparatus according to claim 1 in which the color display material is polyester (Mylar®) coated with thermochromic liquid crystal material that displays color in the approximate temperature range, 20-30 degrees Centigrade.

4. An apparatus according to claim 1 which conforms closely around the lower leg so that the tendon is imaged and is held tightly in place using hook and loop fasteners.

5. An apparatus according to claim 1 which conforms closely around the fetlock joint and is held tightly in place using hook and loop fasteners.

6. An apparatus according to claim 1 which conforms closely around the front of the hock joint and is held tightly in place using hook and loop fasteners.

7. An apparatus according to claim 1 which conforms closely to the back of the animal and is held tightly in place using straps.

8. A method of detecting underlying injury in a mammal patient comprising (a) applying a thermal imaging apparatus to the mammal in an area that is suspected of harboring an injury, (b) applying a thermal imaging apparatus to the mammal in an area that is not suspected of harboring an injury, and comparing the results of (a) and (b).
